(19) United States
(12) Patent Application Publication
(10) Pub. No.: US 2002/0039787 A1
(43) Pub. Date: Apr. 4, 2002

(54) Method for culturing cells

(76) Inventors: Peter Adrian Walton Rogers, Caulfield (AU); Caroline Eve Gargett, Blackburn North (AU); Kristina Bucak, Yarraville (AU)

Correspondence Address: SCULLY, SCOTT, MURPHY & PRESSER, 400 Garden City Plaza, Garden City, NY, 11530, US

(21) Appl. No.: 09/770,956

(22) Filed: Jan. 26, 2001

Related U.S. Application Data
(60) Provisional application No. 60/178,496, filed on Jan. 27, 2000.

(51) Int. Cl. C12N 5/06; C12N 5/08

(52) U.S. Cl. 435/325; 435/366

(57) ABSTRACT

The invention provides a cell culture process and a method for the in vitro growth of microvascular endothelial cells, including myometrial cells and diagnostic, therapeutic and prophylactic applications of microvascular endothelial cells.

### FIELD OF THE INVENTION

The present invention relates generally to a cell culture process and more particularly to a method for the in vitro growth of microvascular endothelial cells. Still more particularly, the present invention provides a method for the in vitro growth of myometrial microvascular endothelial cells. The endothelial cells which are grown in accordance with the method of the present invention are useful in a variety of diagnostic, therapeutic and prophylactic applications such as for use in in vitro angiogenesis assays.

### BACKGROUND OF THE INVENTION

Bibliographic details of the publications referred to by author in this specification are collected at the end of the description.

The microvascular endothelium has a critical role in angiogenesis, the process by which new blood vessels develop from preexisting vessels. In the adult, angiogenesis rarely occurs under normal circumstances, except in the female reproductive tract during the menstrual cycle and pregnancy (Risau, 1997). Little is known about the microvasculature of the myometrium and the changes that occur during pregnancy, where angiogenesis undoubtedly occurs as the myometrial smooth muscle hypertrophies.

The study of the angiogenesis has been significantly advanced by the ability to culture endothelial cells in vitro. Initially large vessel endothelial cells, such as those isolated from the human umbilical vein were used for these studies (Jaffe et al. 1973), but increasingly it has been recognised that microvascular endothelial cells are a more appropriate model since angiogenesis involves microvascular endothelial cells rather than large vessel endothelial cells (Hewett and Murray, 1993; 1993; Bicknell, 1996). It is well known that endothelial cells are heterogeneous in phenotype, function, expression of surface molecules and responsiveness to growth factors (Auerbach et al. 1985; McCarthy et al. 1991; Garlanda and Dejana, 1997). Differences exist between large vessel and microvascular endothelial cells and the properties of arterial and venous endothelial cells are also distinct. Endothelial cells express organ-specific antigens and therefore differ between the various organs, but even within an organ, there is heterogeneity of endothelial cells from different vascular beds (Auerbach et al. 1985; Kumar et al. 1987).

In comparison to large vessel endothelial cells, microvascular endothelial cells are difficult to isolate and culture. Microvascular endothelial cells only comprise 1-5% of the cells in a given tissue, they grow slowly in culture and are contact inhibited. As a result, microvascular endothelial cell cultures are often overgrown by faster growing, non-contact-inhibited fibroblasts, smooth muscle cells and pericytes, usually limiting passage number to five or six (Hewett and Murray, 1993; Bicknell, 1996).

Microvascular endothelial cells have previously been isolated from a number of human tissues including neonatal foreskin, brain, omentum, synovium, retina, endometrium and decidual tissue (Richard et al. 1998; Chung-Welch et al. 1997; Jackson et al. 1990; Su and Gilles, 1992; Iruela-Arispe et al. 1999; Grimwood et al. 1995). There have been few attempts to isolate and culture microvascular endothelial cells from muscular organs and to date such cells have not been successfully cultured. Further, it has not been possible to isolate substantially purified populations of microvascular endothelial cells from any tissue source nor to culture these cells such that sufficient numbers of substantially pure microvascular endothelial cells are generated.

Accordingly, in order to facilitate the design and implementation of applications such as in vitro angiogenesis assays or microvascular endothelial cell grafting, there is a need to develop in vitro culturing systems which support the proliferation and/or differentiation of microvascular endothelial cells, thereby providing a suitable in vitro source of this tissue. In work leading up to the present invention, the inventors have developed a method for the in vitro culturing of microvascular endothelial cells and, in particular, myometrial microvascular endothelial cells.

### SUMMARY OF THE NVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention contemplates a method for the in vitro culturing of microvascular endothelial cells, said method comprising culturing an enriched population of microvascular endothelial cells in the presence of an effective amount of human serum or functional derivative or equivalent thereof for a time and under conditions sufficient to support the growth of said microvascular endothelial cells.

Another aspect of the present invention contemplates a method for the in vitro culturing of microvascular endothelial cells, said method comprising culturing an enriched population of microvascular endothelial cells in the presence of an effective amount of human serum or functional derivative or equivalent thereof and a mitogen or functional derivative or equivalent thereof for a time and under conditions sufficient to support the growth of said microvascular endothelial cells.

Still another aspect of the present invention more particularly provides a method for the in vitro culturing of myometrial microvascular endothelial cells, said method comprising culturing an enriched population of myometrial microvascular endothelial cells in the presence of an effective amount of human serum or functional derivative or equivalent thereof and a mitogen or functional derivative or equivalent thereof for a time and under conditions sufficient to support the growth of said myometrial microvascular endothelial cells.

Yet another aspect of the present invention preferably provides a method for the in vitro culturing of human myometrial microvascular endothelial cells, said method comprising culturing an enriched population of human microvascular endothelial cells in the presence of an effective amount of human serum or functional derivative or equivalent thereof and a mitogen or functional derivative or equivalent thereof for a time and under conditions sufficient to support the growth of said human myometrial microvascular endothelial cells.

Still yet another aspect of the present invention provides a method for the in vitro culturing of microvascular endothelial cells, said method comprising culturing an at least 99% pure population of said microvascular endothelial cells in the presence of an effective amount of human serum or functional derivative or equivalent thereof and a mitogen or functional equivalent or derivative thereof for a time and under conditions sufficient to support the growth of said microvascular endothelial cells.

Preferably, said enriched population of microvascular endothelial cells are obtained by enriching a starting cellular population comprising microvascular endothelial cells, which enrichment comprises the steps of:

(i) selecting for myometrial microvascular endothelial cells from said starting population;

(ii) pre-culturing the cells selected in accordance with step (i) to 60-80% confluence; and

(iii) selecting for microvascular endothelial cells from the 60-80% confluent cell population of step (ii).

A further aspect of the present invention provides a method for the in vitro culturing of microvascular endothelial cell, said method comprising culturing an enriched population of microvascular endothelial cells in the presence of 20% v/v human serum or functional derivative or equivalent thereof and 5 ng/mil bFGF or functional derivative or equivalent thereof for a time and under conditions sufficient to support the proliferation of said microvascular endothelial cells.

Another further aspect of the present invention contemplates a method for the in vitro culturing of microvascular endothelial cells, said method comprising culturing an enriched population of microvascular endothelial cells in the presence of 15% human serum, 5% fetal calf serum or functional derivative or equivalent thereof and an effective amount of either bFGF or VEGF or functional derivative or equivalent thereof for a time and under conditions sufficient to support the proliferation of said microvascular endothelial cells.

Still another further aspect of the present invention contemplates a method for the in vitro culturing of microvascular endothelial cells, said method comprising culturing an enriched population of microvascular endothelial cells in the presence of 15% human serum, 5% fetal calf serum or functional derivative or equivalent thereof, 5 ng/ml bFGF or functional derivative or equivalent thereof and an effective amount of heparin or functional derivative or equivalent thereof for a time and under conditions sufficient to support the proliferation of said microvascular endothelial cells.

Still yet another further aspect of the present invention provides a method for the in vitro culturing of microvascular endothelial cells, said method comprising culturing an enriched population of microvascular endothelial cells in the presence of an effective amount of human serum or functional derivative or equivalent thereof and a mitogen or functional derivative or equivalent thereof for a time and under conditions sufficient to support the growth of said microvascular endothelial cells wherein said culture does not comprise cAMP or functional derivative or equivalent thereof or corticosteroids or functional derivative or equivalent thereof.

In another aspect, the present invention contemplates a method of producing a microvascular endothelial cell culture said method comprising culturing an enriched population of microvascular endothelial cells in the presence of an effective amount of human serum or functional derivative or equivalent thereof and a mitogen or functional derivative or equivalent thereof for a time and under conditions sufficient to support the growth of said microvascular endothelial cells.

Yet another aspect of the present invention contemplates a method of producing a microvascular endothelial cell culture, said method comprising enriching a starting cellular population comprising myometrial microvascular endothelial cells, which enrichment comprises the steps of:

(i) selecting for myometrial microvascular endothelial cells from said starting cellular population;

(ii) culturing the cells selected in accordance with step (i) to 60-80% confluency; and

(iii) selecting for myometrial microvascular endothelial cells from the 60-80% confluent cell population of step (ii)

and culturing said enriched population of said microvascular endothelial cells in the presence of an effective amount of human serum or functional derivative or equivalent thereof and a mitogen or functional derivative or equivalent thereof for a time and under conditions sufficient to support the growth of said human myometrial microvascular endothelial cells.

A further aspect of the present invention contemplates the use of an enriched population of microvascular endothelial cells, an effective amount of human senum or functional derivative or equivalent thereof and an effective amount of mitogen or functional derivative or equivalent thereof in the manufacture of a microvascular endothelial cell culture which is capable of supporting the growth of said microvascular endothelial cells.

Yet another aspect of the present invention is directed to the use of the microvascular endothelial cells generated in accordance with the method of the present invention in the manufacture of a medicament for the prophylactic or therapeutic treatment of a manual.

The present invention should also be understood to extend to the use of microvascular endothelial cells generated in accordance with the method of the present invention in any diagnostic, experimental, prophylactic or therapeutic procedure. In this regard, reference to “experimental” includes reference to the use of the subject cells in screening assays designed to identify molecules which modulate angiogenesis or any other functional activity of the subject cells.

In still another aspect, the present invention is directed to microvascular endothelial cells generated in accordance with the method of the present invention, which cells have been immortalised or otherwise transformed or transfected, and to the use of such cells.

### BRIEF DESCRIPTION OF THE DRAWINGS

FIG. 1 is a photographic representation of human myometrial microvascular endothelial cells. Imnmunolocalisation of Ulex europeaus antigen 1 (UEA) to microvascular endothelium of normal human myometrium (A) and cultured myometrial microvascular endothelial cells (B) passage 1 (P1), (C) passage 5 (P5) and (D) passage 14. Note that some P1 MEC have attached Dynabeads. MEC (P5) express (E) Factor VIII related antigen (FVIIIra) shown by periuclear granular staining of the Weibel-Palade bodies, (F) CD31 in the perinuclear region and at sites of cell-cell contact, but not (G) α-smooth muscle actin as shown by the lack of background staining, in contrast to the single contaminating smooth muscle cell on this coverslip, which contained approximately 1.7×10⁴ MEC. Negative control (H) for FVIIIra. Similar negative controls were obtained for CD31 and UEA-1 (results not shown). Scale bars: 50 μm.

FIG. 2 is a photographic representation of cultured human myometrial microvascular endothelial cells. Phase contrast micrographs of confluent monolayer cultures of (A) primary cultures of (B) late passage (P11) MEC showing typical cobblestone morphology and (C) the formation of capillary tube-like structures when cultured on Matrigel. (D) Fluorescence micrograph of MEC labelled with Dil-Ac-LDL after 4 hours incubation. Scale bars: 50 μm.

FIG. 3 is a graphical representation of the effect of serum on proliferation of myometrial microvascular endothelial cells using the MTS bioassay. MEC (P5) were incubated (A) without (◯,□) or with 5 nglml bFGF (●,▪) for 5 days in M199 culture medium containing human serum (HS) (●,◯) or fetal calf serum (FCS) (□,▪) and (B) in M199 culture medium containing 5 ng/ml bFGF and 20% HS (Δ), 20% PCS (▪), 15% HS/5% FCS (●), 10% HS/10% FCS (▴), 5% HS/15% FCS (⋄). Results shown are means ± SEM of triplicates from a single experiment, representative of 3.

FIG. 4 is a graphical representation of the proliferation of myometrial microvascular endothelial cells in the presence and absence of cAMP promoters. MEC (P5) were incubated in M199 medium containing 15%HS/5% FCS and 0.1 mg/ml heparin without (▪) or with (●,♦,▴) 5 ng/rnl bFGF in the presence of IBMX (♦) or DCM (▴) and absorbance measured as indicated. Results shown are means ± SEM of triplicates from a single experiment. A similar result was obtained with P2 MEC.

FIG. 5 is a graphical representation of the growth response curves of myometrial microvascular endothelial cells stimulated with maximal concentrations of growth factors. Quiescent P5 MEC were incubated in M199 culture medium containing 20% HS in the absence (●) or presence of (▪) of 120 μg/ml endothelial cell growth supplement ECGS, (□) 20 μg/ml endothelial cell growth factor (ECGF), (⋄) 10 ng/ml bFGF, two isoforms of vascular endothelial growth factor (VEGF), VEGF₁₂₁ (▴), VEGF₁₆₅ (Δ), each 10 ng/ml and (◯) 50 ng/ml epidermal growth factor and absorbance measured as indicated. Results shown are means ± SEM from a single experiment, representative of 3.

FIG. 6 is a graphical representation of the growth response curves of VEGF- and bFGF- induced myometrial microvascular endothelial cell proliferation. Quiescent low passage (P1, P2, and P3) (●) and high passage MEC (P10 and P13) (◯) were incubated with either (A) VEGF or (B) bFGF for 6 days and absorbance measured. Data are changes in absorbance over 6 days expressed as percentage of maximal response for each growth factor and cell type. Shown are means ± SEM of 3 experiments for low passage MEC and means for 2 experiments for high passage MEC, which varied less than 10% and 18% for VEGF and bFGF respectively.

FIG. 7 is a photomicrograph showing myometrial microvascular endothelial cell migration. Monolayers of confluent MEC (P3) were wounded and migration of MEC examined after 24 hours incubation in basal M199 medium and (A) 5 ng/ml bFGF or (B) 5 NG/ML bFGF and 50 μg/ml anti-human bFGF IgG. Note that some MEC have dynabeads attached. Scale bars: 100 μm.

### DETAILED DESCRIPTION OF THE INVENTION

The present invention is predicated, in part, on the development of a method for the in vitro growth of microvascular endothelial cells and, in particular, myometrial microvascular endothelial cells. In this regard, the inventors have determined that the culturing of an enriched source of microvascular endothelial cells in a minimal culture medium comprising human serum and a mitogen provides the essential growth requirement for stimulation of in vitro microvascular endothelial cell proliferation. Without limiting the present invention in any way, microvascular endothelial cells cultured in accordance with the method of the present invention can be passaged for longer, and thereby larger cell numbers generated, than cells which are isolated and cultured in accordance with prior art methods.

Accordingly, one aspect of the present invention contemplates a method for the in vitro culturing of microvascular endothelial cells, said method comprising culturing an enriched population of microvascular endothelial cells in the presence of an effective amount of human serum or functional derivative or equivalent thereof for a time and under conditions sufficient to support the growth of said microvascular endothelial cells.

More particularly, the present inventors contemplate a method for the in vitro culturing of microvascular endothelial cells, said method comprising culturing an enriched population of microvascular endothelial cells in the presence of an effective amount of human serum or functional derivative or equivalent thereof and a mitogen or functional derivative or equivalent thereof for a time and under conditions sufficient to support the growth of said microvascular endothelial cells.

Reference to “microvascular endothelial cells” should be understood as a reference to cells exhibiting microvascular endothelial cell morphology, phenotype and/or functional activity and to mutants or variants thereof. Said microvascular endothelial cells may be at any differentative stage of development. “Variants” include, but are not limited, cells exhibiting some but not all of the morphological or phenotypic features or functional activities of microvascular endothelial cells at any differentative stage of development. “Mutants” include, but are not limited to, microvascular endothelial cells which are transgenic wherein said transgenic cells are engineered to express one or more genes such as genes encoding specific antigens, cytokines or receptors.

It should be further understood that the microvascular endothelial cells which are used in accordance with the method of the present invention may be either freshly harvested (for example, via a biopsy specimen) or may have been harvested at an earlier time point and subsequently stored or cultured. For example, said microvascular endothelial cells may be derived from frozen stocks or may have been transiently maintained in in vitro culture conditions which maintained the cells in a viable state. Examples of microvascular eadothelial cells suitable for use in the method of the present invention include, but are not limited to, microvascular endothelial cells isolated from neonatal foreskin, brain, omentum, synovium, retina, myometrium, endometrium, tumours (malignant or benign) which comprise microvascular endothelial cells (such as fibroids), adipose tissue and decidual tissue with respect to tumours, microvascular endothelial cells isolated from malignant tissue have been difficult to grow utilising prior art methods while the successful in vitro culturing of microvascular endothelial cells from benign tumours has not been possible. The method of the present invention has overcome the prior art problems and can be utilised to culture tumour derived microvascular endothelial cells. Preferably, said microvascular endothelial cells are isolated from muscular organs and even more preferably from the myometrium.

Accordingly, the present invention more particularly provides a method for the in vitro culturing of myometrial microvascular endothelial cells, said method comprising culturing an enriched population of myometrial microvascular endothelial cells in the presence of an effective amount of human serum or functional derivative or equivalent thereof and a mitogen or functional derivative or equivalent thereof for a time and under conditions sufficient to support the growth of said myometrial microvascular endothelial cells.

The morphological and functional features of microvascular endothelial cells may include any one or more features characteristic of microvascular endothelial cells. For example, this includes but is not limited to the capacity to bind UEA-1 lectin, the expression of CD31, the expression of factor VIII related antigen in Weibel-Palade bodies in the perinuclear region and at sites of cell-cell contact, and the development of cobblestone morphology. The subject cells may also exhibit the capacity to take up DiI-Ac-LDL via the scavenger LDL receptor. In terms of the functional activities of these cells, they may also exhibit the capacity to form capillay-like tubes on a basement membrane matrix and to migrate and proliferate in response to bFGF or VEGF. Microvascular endothelial cells typically do not express cytokeratin or the smooth muscle cell marker-α-smooth muscle actin.

The microvascular endothelial cells of the present invention may be derived from any animal including humans, primates, livestock animals (eg. horses, cattle, sheep, pigs, donkeys), laboratory test animals (eg. mice, rabbits, rats, guinea pigs), companion animals (eg. dogs, cats), captive wild animals (eg. kangaroos, deer, foxes), aves or reptiles. Preferably, the mammal is a human or laboratory test animal. Even more preferably the mammal is a human.

The present invention therefore preferably provides a method for the in vitro culturing of human myometrial microvascular endothelial cells, said method comprising culturing an enriched population of human microvascular endothelial cells in the presence of an effective amount of human serum or functional derivative or equivalent thereof and a mitogen or functional derivative or equivalent thereof for a time and under conditions sufficient to support the growth of said human myometrial microvascular endothelial cells.

The microvascular cells which are grown in accordance with the method of the present invention are enriched. By “enriched” is meant that the subject microvascular endothelial cells comprise at least 98%, and more preferably at least 99%, of the in vitro culture cellular population which is ultimately plated. The inventors have determined that in addition to culturing a microvascular endothelial cell population in the minimal media conditions herein disclosed, the cell culture population which is ultimately plated must be at least 98%, and more preferably 99%, pure.

The present invention therefore preferably provides a method for the in vitro culturing of microvascular endothelial cells, said method comprising culturing an at least 99% pure population of said microvascular endothelial cells in the presence of an effective amount of human serum or functional derivative or equivalent thereof and a mitogen or functional equivalent or derivative thereof for a time and under conditions sufficient to support the growth of said microvascular endothelial cells.

Preferably said rnicrovascular endothelial cells are myometrial endothelial cells and even more preferably human myometrial microvascular endothelial cells.

Although the requisite degree of enrichment can be achieved by any suitable method, to date the prior art methods have only achieved enrichment of 90-95%. However, the inventors have developed an enrichment protocol which achieves at least 98% purity and up to 99.95% purity Specifically, the inventors have determined that a two step positive selection protocol is effective for achieving the requisite level of purity. The microvascular endothelial cells are first positively selected while in a single cell suspension prior to culturing in the minimal medium herein described. These positively selected cells are then pre-cultured until approximately 75-80% confluence is reached. Reference to “pre-culture” should be understood as a reference to the culturing step which forms part of the enrichment process and should not be cofined with reference to the culturing step which is ultimately performed in order to generate significantly larger numbers of substantially more pure microvascular endothelial cells than has been available utilising prior art methods. It should be understood that a “pre-culture” step is one which, in accordance with the preferred method disclosed herein is to be followed by a selection procedure prior to setting up the ultimate cultures. Any suitable cell culture medium may be utilised for the subject pre-cultures although use of the minimal medium herein disclosed is preferred. Following pre-culturing of the positively selected microvascular endothelial cells to approximately 75-80% confluence, the pre-cultured cells are once more rendered a single cell suspension and a further positive selection step is performed. This enrichment protocol has been demonstrated to produce a final population of microvascular endothelial cells of greater than 99% purity. It should be understood, however, that pre-culcuring of the positively selected cells to 75-80% confluency is a preferred embodiment only and a further selection step can, in fact, be performed on microvascular endothelial cells which have been pre-cultured to approximately 50-100% confluency and preferably to 60-80% confluency. In assessing the degree of confluency, regard may be had to the approximate percentage confluency of the cells when the population of cells are examined as a whole relative to the surface area of the culture dish when considered as a whole. However, the subject microvascular endothelial cells, may also seed and divide such that clusters of cells are formed which clusters are spread across the culture dish. “Confluency” may also be considered in terms of the approximate percentage confluency of cells in a given region of the culture dish, which region comprises a dividing cluster of cells.

Enrichment by “positive selection” should be understood as the process of identifying and then separating, from a starting population of cells, the cells of interest. In this regard, reference to “starting” population of microvascular endothelial cells should be understood to mean the population of cells comprising microvascular endothelial cells which is to be the subject of enrichment and culturing in order to produce an in vitro population of microvascular endothelial cells. As detailed hereafter, the starting population may be derived from any suitable source such as a biopsy. Any suitable positive selection technique may be utilised and would be known to those of skill in the art. For example, the UEA-1-coated dynabead selection method described by Jackson et al (1990) may be utilised both at the initial starting population stage and during early passage of the pre-cultured microvascular endothelial cells. It has been determined that the attached dynabeads do not interfere with microvascular endothelial cell growth or survival and are diluted out within one or two passages.

It should be understood that the application of a positive selection step will only be necessary where the starting population of microvascular endothelial cells is less than 99% pure. This will occur, for example, where the starting population is derived from a biopsy specimen which comprises a heterogeneous population of cells. For example, a myometrial biopsy cell will comprise numerous cell types including the subject microvascular endothelial cells, fibroblasts and smooth muscle cells.

However, to the extent that the subject microvascular endothelial cells are isolated from a homogeneous cell source such as a microvascular endothelial cell tumour or a microvascular cell line, it may not be necessary to perform an enrichment step since the cells may already form an “enrichmed” population in accordance with the method of the present invention.

Without limiting the present invention to any one theory or mode of action, the inventors have determined that a single cell suspension prepared from a starting cell population comprising microvascular endothelial cells will nevertheless evidence a certain amount of cell-cell adherence (often referred to as “clumping”) of the suspended cells. This occurs, for example, where “sticky” cells (i.e. highly adherent cells) such as fibroblasts, pericytes or vascular smooth muscle cells adhere to another cell which is located in close proximity. Although such adherence can be minimised by the presence of certain enzymes or additives in the solution in which the cells are suspended, this phenomena cannot, to date, be eliminated.

Where a fibroblast or smooth muscle cell adheres to a microvascular endothelial cell, the positive selection of that microvascular endothelial cell will necessarily also select for the contaminating non-microvascular cell which has adhered to it. In culturing such a population of positively selected cells, although the proportion of contaminating cells will have been reduced, they will not have been eliminated. In fact, prior art methods have only been able to achieve purity levels of 90-95%. Due to the presence of such contaminating cells, in vitro growth of the subject microvascular cells is either eliminated or severely compromised due to rapid proliferation of the contaminating cells.

The inventors have determined, though, that in a single cell suspension which is prepared from cells which have been cultured in vitro, the incidence of cell-cell adherence is reduced. Accordingly, the application of a farther positive selection step has been found to improve the purity of the subject microvascular endothelial cell population. Specifically, the inventors have demonstrated that the application of a second selection step, such as a positive selection step, to a single cell suspension prepared from a 75-80% confluent in vitro pre-culture of single-step purified microvascular endothelial cells will achieve purity of in excess of 99%. Still without limiting the present invention in any way, it is the combination of achieving a very high purity of microvascular endothelial cells together with the minimal media in vitro culturing conditions herein disclosed which has permitted the inventors to obtain, via in vitro proliferation, significantly larger numbers of microvascular endothelial cells which are of higher purity than has been obtainable utilising prior art culturing. The inventors have shown that non-immortalised microvascular cells which are cultured in accordance with the method disclosed herein can be passaged in excess of 14 times whereas prior art culturing methods have only typically ever achieved 5-6 passages.

As detailed previously, enrichment is preferably performed as a two step positive selection technique, the first positive selection step being applied to a single cell suspension starting population comprising the cells of interest and the second step being applied to the 50-100%, and preferably 75-80%, confluent cell population pre-cultured from the cells positively selected at the first step. To the extent that the initial single cell suspension is prepared from a biopsy or other tissue sample, it may also be necessary to initially digest the tissue sample in order to facilitate the preparation of a single cell suspension. Preferably the digestion is enzymatic digestion. Methods of enzymatically digesting tissue samples will be known to those of skill in the art. Even more preferably, the digestion is performed by dissociating he subject tissue sample sequentially with collogenase and trypsin.

The present invention therefore preferably provides a method for the in vitro culturing of microvascular endothelial cells, said method comprising culturing an at least 99% pure population of said microvascular endothelial cells in the presence of an effective amount of human serum or functional derivative or equivalent thereof and a mitogen or functional equivalent or derivative thereof for a time and under conditions sufficient to support the growth of said microvascular endothelial cells.

Even more preferably, said microvascular endothelial cells are myometrial microvascular endothelial cells and even more preferably human myometrial microvascular endothelial cells.

Reference to “growth” should be understood as a reference to the induction, up-regulation or other enhancement of the proliferation, differentiation and/or functional activity of said microvascular endothelial cells. Preferably, said growth is proliferation.

Accordingly, the present invention preferably provides a method for the in vitro culturing of microvascular endothelial cells, said method comprising culturing an enriched population of microvascular endothelial cells in the presence of an effective amount of human serum or functional derivative or equivalent thereof and a mitogen or functional derivative or equivalent thereof for a time and under conditions sufficient to support the proliferation of said microvascular endothelial cells.

Preferably, said enriched population of microvascular endothelial cells are obtained by enriching a starting cellular population comprising microvascular endothelial cells, which enrichment comprises the steps of:

(i) selecting for myometrial microvascular endothelial cells from said starting population;

(ii) pre-culturing the cells selected in accordance with step (i) to 60-80% confluence; and

(iii) selecting for microvascular endothelial cells from the 60-80% confluent cell population of step (ii).

More preferably, said microvascular endothelial cells are of myometrial origin and even more preferably of human myometrial origin.

Still more preferably said starting cellular population comprising myometrial microvascular endothelial cells is a population of enzymatically digested, tissue sample derived cells.

Reference to “human serunm” should be understood as a reference to fractionated, unfractionated, purified or unpurified human serum or functional derivative or equivalent thereof. The serum may be fractionated, for example, to either concentrate functionally active components or to remove any one or more components which may not significantly contribute to supporting the growth of the microvascular endothelial cells according to the method of the present invention. It should also be understood that the human senun may form a functional component of a culture additive such as plasma. The human serum which is used in the method of the present invention may be naturally derived or may comprise all or some synthetically or recombinantly produced components.

Reference to a “mitogen” should be understood as a reference to a molecule or functional derivative or equivalent thereof which can induce a microvascular endothelial cell to proliferate and/or differentiate. The subject mitogen may be in pure form or it may form a functional component of a more complex composition. The mitogen which is used in the method of the present invention may be naturally derived or may be synthetically or recombinantly produced. Examples of mitogens suitable for use in the present invention include, but are not limited to bFGF, VEFG_{121,} VEGF_{124,} and ECGS or functional derivative or equivalent thereof. Without limiting the present invention in any way, although these mitogens can stimulate microvascular endothelial cell proliferation, these mitogens when alone cannot induce acceptable levels of proliferation. Preferably, said mitogen is bFGF or VEGF and even more preferably bFGF.

In this regard, reference to “functional equivalent or derivative” of said human serum, said human serum component or said mitogen includes, but is not limited to, fragments, said fragments having the functional activity of said component or homologues, analogues, mutants, variants and derivatives thereof. This includes homologues, analogues, mutants, variants and derivatives derived from natural, recombinant or synthetic sources including fusion proteins. Reference to “homologues” should be understood as a reference to the subject component derived from species other than the species from which the component is originally derived.

Derivatives include fragments, parts, portions, mutants, variants and mimetics from natural, synthetic or recombinant sources including fusion proteins. Parts or fragments include, for example, active regions of the subject component. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Chemical and functional equivalents of the subject components should be understood as molecules exhibiting any one or more of the functional activities of these components and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Analogues contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide. Sulphydryl groups may be modified by methods such as carboxymethylation with iodoacetic acid or iodoacemmide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| antinoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-y-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycinle | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylanine | Dmphe | N-(2-carboxyethyl)glycine | Nglu |
| D-α-methylproline | Dmpro | N-(carboxymethy)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillaniine | Nmpen |
| γ-aminobutyric acid | Gabu | N-(p-hydroxyphenyl)glycine | Nhtyr |
| L-t-butylglycine | Thug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylomithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Cross can be used, for example, to stabilise 3D conformations, using homo-bifunctional crosslinkers such as the bifunctional imido esters and having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

Without limiting the present invention to any one theory or mode of action, primary cultures of myometrial microvascular endothelial cells are known to show growth rates which differ between isolates and which does not relate to the age of donor or hormonal status. A feature common to all isolates, however, is the requirement for human serum and a mitogen or functional derivative or equivalent thereof to establish and maintain myometrial microvascular endothelial cell growth. Preferably said human serum is used at a concentration of 20% v/v and said bFGF, in a most preferred embodiment, is used at 5 ng/ml. This requirement remains necessary through all passages until senescence is reached. Without limiting the present invention in any way, senescence has been observed to occur at around the 17-20th passage.

The present invention therefore preferably provides a method for the in vitro culturing of microvascular endothelial cell, said method comprising culturing an enriched population of microvascular endothelial cells in the presence of 20% v/v human serun or functional derivative or equivalent thereof and 5 ng/ml bFGF or functional derivative or equivalent thereof for a time and under conditions sufficient to support the proliferation of said microvascular endothelial cells

Preferably said microvascular endothelial cells are of myometrial origin and more preferably of human myometrial origin.

It should be understood that the culture system of the present invention may comprise human serum and a mitogen as the only source of growth stimulant or it may comprise any one or more additional components which provide a growth stimulus or otherwise provide some form of direct or indirect support to the cells which are cultured in accordance with the method of the present invention, Said “support” may be by way of providing, for example, components which are required for the cell to metabolise (eg. glutamine), components which buffer the cell culture medium in order to assist in maintaining a particular pH range, heparin, or antibiotics to maintain the sterility of the culture. Accordingly, the human serum and mitogen may be co-admrinistered to the culture with one or more other component. The base culture medium to which the human serum, mitogen or other additives are administered may be any suitable medium such as, but not limited to the commercially available M199, DME or RPMI. Preferably the base medium is M199.

By “co-administered” is meant either simultaneous administration of the components and human serum or their sequential administration. By “sequential” administration is meant a time difference of from seconds, minutes, hours or days between the administration of tile various components. These components may be administered in any order. For example, myometrial microvascular endothelial cells proliferate poorly in fetal calf serum. However, acceptable rates of growth are observed where both fetal calf serum and human serum are used in combination. This indicates that fetal calf serum may comprise a component which functions synergistically with one or more components present in human serum to enhance microvascular endothelial cell growth.

In another preferred embodiment, the method of the present invention utilises human serum together with fetal calf serum in a ratio of not less than 3:1 and an effective amount of either bFGF or VEGF or functional derivative or equivalents thereof.

According to this preferred embodiment, the present invention contemplates a method for the in vitro culturing of microvascular endothelial cells, said method comprising culturing an enriched population of microvascular endothelial cells in the presence of 15% human serum, 5% fetal calf serum or functional derivative or equivalent thereof and an effective amount of either bPGF or VEGF or functional derivative or equivalent thereof for a time and under conditions sufficient to support the proliferation of said microvascular endothelial cells.

Even more preferably, the present invention contemplates a method for the in vitro culturing of microvascular endothelial cells, said method comprising culturing an enriched population of microvascular endothelial cells in the presence of 15% human serum, 5% fetal calf serum or functional derivative or equivalent thereof, 5 ng/ml bFGF or functional derivative or equivalent thereof and an effective amount of heparin or functional derivative or equivalent thereof for a time and under conditions sufficient to support the proliferation of said microvascular endothelial cells.

Reference to an “effective” amount is a reference to an amount necessary to at least partly achieve the desired outcome.

Preferably said heparin is utilised at a concentration of 0.1 mg/ml.

More preferably said microvascular cells are of myometrial origin and still more preferably of human myometrial origin.

In still another preferred embodiment, the enriched population of myometrial microvascular endothelial cells which are utilised in accordance with these preferred culture conditions are obtained by enriching a starting cellular population comprising myometrial microvascular endothelial cells, which enrichment comprises the steps of:

(i) selecting for myometrial microvascular endothelial cells from said starting cellular population;

(ii) pre-culturing the cells selected in accordance with step (i) to 60-80% confluency; and

(iii) selecting for myometrial microvascular endothelial cells from the 60-80% confluent cell population of step (ii).

Still more preferably said starting population of myometrial microvascular endothelial cells is a population of enzymatically digested, tissue sample derived cells.

In a related aspect, the inventors have shown that the presence of cAMP promoters was not required as a component of tie growth medium in which the subject microvascular endothelial cells are cultured. This is of particular use since the presence of cAMP promoters in growth medium complicates any investigation examining receptor mediated functions that signal through adenylyl cyclase. In addition, cAMP promoters may also interact with other signalling pathways, including growth factor receptors with intrinsic tyrosine kinase activity. The inventors have also determined that it is not necessary to include corticosteroids in the culture medium utilised herein.

Accordingly, in a related aspect the present invention provides a method for the in vitro culturing of microvascular endothelial cells, said method comprising culturing an enriched population of microvascular endothelial cells in the presence of an effective amount of human serum or functional derivative or equivalent thereof and a mitogen or functional derivative or equivalent thereof for a time and under conditions sufficient to support the growth of said microvascular endothelial cells wherein said culture does not comprise cAMP or functional derivative or equivalent thereof or corticosteroids or functional derivative or equivalent thereof.

Preferably, said microvascular endothelial cells are myometrial microvascular endothelial cells and even more preferably said growth is proliferation.

In another aspect, the present invention contemplates a method of producing a microvascular endothelial cell culture said method comprising culturing an enriched population of microvascular endothelial cells in the presence of an effective amount of human serum or functional derivative or equivalent thereof and a mitogen or functional derivative or equivalent thereof for a time and under conditions sufficient to support the growth of said microvascular endothelial cells.

Preferably, the method of the present invention contemplates a method of producing a microvascular endothelial cell culture, said method comprising enriching a starting cellular population comprising myometrial microvascular endothelial cells, which enrichment comprises the steps of:

(i) selecting for myometrial microvascular endothelial cells from said starting cellular population;

(ii) culturing the cells selected in accordance with step (i) to 60-80% confluency; and

(iii) selecting for myometrial microvascular endothelial cells from the 60-80% confluent cell population of step (ii)

and culturing said enriched population of said microvascular endothelial cells in the presence of an effective amount of human serum or functional derivative or equivalent thereof and a mitogen or functional derivative or equivalent thereof for a time and under conditions sufficient to support the growth of said human myometrial microvascular endothelial cells.

Still more preferably said starting population of myometrial microvascular endothelial cells is a population of enzymatically digested, tissue sample derived cells. Even more preferably said human serum is used at a concentration of 20% v/v and said mitogen is either 5 ng/ml bFGF or VEGF or functional derivative or equivalent thereof.

In another preferred embodiment said human serum is used at a concentration of 15% v/v together with 5% v/v foetal calf serum and said mitogen is 5 ng/ml bFGF or VEGF or functional derivative or equivalent thereof.

Preferably, said microvascular endothelial cells are myometrial microvascular endothelial cells and said growth is proliferation.

A further aspect of the present invention contemplates the use of an enriched population of microvascular endothelial cells, an effective amount of human serum or functional derivative or equivalent thereof and an effective amount of mitogen or functional derivative or equivalent thereof in the manufacture of a microvascular endothelial cell culture which is capable of supporting the growth of said microvascular endothelial cells.

Preferably, said microvascular endothelial cells are myometrial microvascular endothelial cells and even more preferably human myometrial microvascular endothelial cells. Still more preferably, said growth is proliferation.

Yet more preferably, said enriched population of myometrial microvascular endothelial cells are obtained by enriching a starting cellular population comprising myometrial microvascular endothelial cells, which enrichment comprises the steps of:

(i) selecting for myometrial microvascular endothelial cells from said starting cellular population;

(ii) culturing the cells selected in accordance with step (i) to 60-80% confluency; and

(iii) selecting for myometrial microvascular endothelial cells from the 60-80% confluent cell population of step (ii).

Still more preferably said starting population of myometrial microvascular endothelial cells is a population of enzymatically digested, tissue sample derived cells.

The methods and compositions of the present invention are useful for generating microvascular endothelial cells for use in a range of therapeutic, prophylactic and diagnostic procedures. For example, the cells produced by the method of the present invention can be used:

for in vitro angiogenesis assays.

to assess the effects of growth factor or endothelial cell functions and angiogenesis.

as targets for immobilization by standard techniques.

to screen for inhibitors and/or agonists of angiogenesis.

as a gene therapy tool. For example, the cells may be genetically modified and then returned to a subject.

to endothelialise graft tissue. This may be achieved for example, by intravenously injecting the subject cells into a graft.

for endothelial cell signalling studies.

for use in the analysis of endothelial cell biology.

as a transplant.

To the extent that the microvascular endothelial cells generated in accordance with the method of the present invention are administered to a subject, their administration may be performed in a syngeneic, allogeneic or xeaogeneic fashion. Preferably, the cells are administered syngeneically, such as would occur if a sample of microvascular endothelial cells were removed from a subject, cultured in vitro in accordance with the method of the present invention and then returned to the same subject. This may occur, for example, where a subject has been the recipient of a graft and a sample of the subject's microvascular endothelial cells were removed and cultured in accordance with the method of the present invention for the purpose of providing re-vascularization therapy to the graft of said subject.

Accordingly, yet another aspect of the present invention is directed to the use of the microvascular endothelial cells generated in accordance with the method of the present invention in the manufacture of a medicament for the prophylactic or therapeutic treatment of a mammal.

The present invention should also be understood to extend to the use of microvascular endothelial cells generated in accordance with the method of the present invention in any diagnostic, experimental, prophylactic or therapeutic procedure. In this regard, reference to “experimental” includes reference to the use of the subject cells in screening assays designed to identify molecules which modulate angiogenesis or any other functional activity of the subject cells.

In still another aspect, the present invention is directed to microvascular endothelial cells generated in accordance with the method of the present invention, which cells have been immortalised or otherwise transformed or transfected, and to the use of such cells. It should be understood that not all transformed or transfected cells will be immortalised since such transformations or transfections may be transient in nature. The present invention should be understood to extend to the subject cells which have undergone any form of transformation or transfection.

The present invention is further described by the further non-limiting Examples.

### EXAMPLE 1

### Human Tissues

Human myometrial tissue was obtained from normal ovulating women undergoing hysterectomy (aged 20-40 years) without underlying uterine pathology, usually for prolapse. In some cases, myometrium was obtained from women undergoing hysterectomy for fibroids. Tissues included in this study were from women who had not taken exogenous hormones in the previous three months. Informed consent was obtained from each patient and ethical approval from the Monash Medical Centre Human Research and Ethics Committee.

A small portion of each piece of tissue was fixed for 4 hours in 10% phosphate-buffered formalin (pH 7.4) for routine paraffin embedding. Sections (5 μm) were used for immunohistochemical analysis. The remaining tissue (2 -7 g) was collected in HEPES-buffered M199 culture medium (Gibco BRL, Gaithersburg, Md., USA) containing 10% FCS and antibiotic-antimycotic solution (Gibco, BRL) with final concentrations of penicillin 1000 U/ml, streptomycin 1000 U/ml and fungizone 2.5 μg/ml. The tissue was stored overnight at 4° C. and then processed.

### EXAMPLE 2

### Microvascular Endothelial Cell Isolation and Purification

The endometrial layer of the uterine tissue was identified, removed and discarded together with the first 1 mm of myometrial tissue and the outer third of the myometrium, thus removing any of the serosa and mesothelial layer. The inner ⅔ of the myometrium was then finely chopped for enzyme dissociation, since the inner layers of myometrium show greater responses to sex steroid hormones compared to the outer third (Noe et al., 1999). Chopped tissue was digested with 2 mg/ml collagenase type 2 (Worthington, Biochemical Corporation, Freehold, N.J., USA) and 14.5 μg/ml deoxyribonuclease type I (Boehringer Mannheim GmbH, Mannheim, Germany) in CA²⁺- and Mg²⁺-free phosphate buffered saline (PBS, pH 7.4) containing 0.1% BSA (Sigma Chemical Co., St. Louis, Mo., USA) for 2 hour at 37° C. in a shaking water bath. The cloudy supernatant containing single cells was sequentially removed at 30 minute intervals during the dissociation procedure and placed into HEPES-buffered M199 medium containing 50% FCS and stored at 4° C. until all the tissue was dissociated. Fresh enzyme solution was added back to the digesting tissue. The cell suspensions were collected, diluted in M199 medium and filtered through 100 μm stainless steel mesh (Sigma), washed several times and incubated with trypsin:EDTA (0.05% trypsin: 0.53 mM EDTA, Gibco BRL) and 24 μg/ml deoxyribonuclease type I for 10 minutes at 37° C. to digest the remaining microvessel fragments and obtain single cell suspensions. The cells were washed and resuspended at 2.5×10⁷/ml in HEPES-buffered M199 medium containing 1% FCS. Cell viability after collagenase and trypsin treatment was approximately 70 and 60% respectively.

Dynal M-450 magnetic beads (Dynal, Oslo, Norway) were coated with lectin by incubating 2×10⁷ beads with UEA-1(1 mg/ml in 0.1 M borate buffer, pH 8.5) (Sigma) in a 200 μl volume 24 hour at 4° C., according to the manufacturer's instructions. The beads were collected using a magnetic particle concentrator (MPC; Dynal) and washed 3 times in PBS/0.1%BSA (one an overnight wash) and resuspended at 2×10⁸/ml in PBS/ISA.

Endothelial cells were positively selected from the dissociated tissue by incubating 1 ml volumes of cell suspension (2.5×10⁷ cells) with 8×10⁷ UEA-1 coated beads for 10 minutes at 4° C. with end-over-end rotation (bead:EC ratio ≈5:1). The bead-attached cells were recovered and washed 8-10 times in M199/1%FCS using the MPC.

### EXAMPLE 3

### Microvascular Endothelial Cell Culture

The purified MEC were resuspended in a standard culture medium comprising M199 with Earle's salts containing heat-inactivated 15% male human serum (HS) (obtained from Red Cross Blood Service and male staff volunteers) and 5% FCS (CSL, Melbourne, Australia), 2 mM glutamine (Gibco BPL), 5 ng/ml bFGF (Gibco BRL), 0.1 mg/ml heparin (Gibco BRL), and antibiotic/antimycotic, seeded into culture flasks at 8-10×10⁴ cells/cm² coated with 10 μg/ml fibronectin (Gibco BRL) and incubated in a humidified atmosphere at 37° C. in 5% CO₂ in air. For some isolations 0.5 μg/ml hydrocortisone (Sigma), 330 μM 3-isobutyl-1-methyl xanthine (IBMX: Sigma), 2 mM MgSO⁴ (complex culture medium) were also included in the culture medium. Medium was changed every 2-3 days and at 70-80% confluence, MEC were trsinised (0.025% trypsin: 0.27 mM EDTA) and repurified with UEA-1 coated dynabeads and subcultured at a split ratio of 1:3 on 0.2% gelatin-coated (Sigma) T175 Falcon tissue culture flasks (Beckton Dickinson, Bedford Mass., USA). On some occasions it was necessary to remove contaminating smooth muscle cells with a further dynabead purification at a later passage. For freezing, MEC (5×10⁶-2×10⁷) were resuspended in 20% FCS/10%DMSO (Sigma/70% culture medium and slowly cooled to −80° C. and then stored in liquid N₂. Thawed cells were gently washed in culture medium and seeded at 1.5×10⁴/cm² on fibronectin-coated flasks.

### EXAMPLE 4

### Immunhistochemistry

MEC were grown on 13 mm gelatin-coated Thermanox coverslips (Nunc Roskilde, Denmark) and when confluent were rinsed in PBS and fixed in cold acetone for 2 minutes. Standard immunohistochemistry protocols (Abberton et al. 1999; Goodger (MacPherson) and Rogers, 1994; Gargett et al. 1999) were used after first blocking sections with 0.03% H₂O₂ for 10 minutes at room temperature (RT) and protein blocking reagent (PBA) (Lipshaw Immunon, Pittsburgh, Pa., USA) for 10 minutes at RT. The primary antibodies were then incubated for 1 hour at 37° C., followed by biotinylated rabbit anti-mouse or goat anti-rabbit secondary antibodies (1/100) for 30 minutes at RT, streptavidin-HRP conjugate (Zymed, San Francisco, Calif., USA) for 30 minutes at RT and AEC chromagen (Zymed) for 10 minutes. The following primary antibodies were used: rabbit anti-human Factor VIII related antigen at 20 μg/ml (Zymed), mouse anti-human CD31, 8.2 μg/mnl (Zymed), mouse anti-human CD34, 0.1 μg/ml (QBEND/10 clone; Serotec, Oxford, UK), mouse anti-hunan cc smooth muscle actin, 0.18 μg/ml (Dako Ltd, High Wycombe, UK), mouse anti-human cytokeratin, 2 μg/ml (Clone MNF116; Dako). Ulex europeaus A-1 binding antigen was detected on MEC grown on coverslips and in tissue sections of myometrium using 20 μg/ml biotinylated UEA-1 lectin (Sigma) for 30 minutes at room temperature, followed by streptavidin-HRP conjugate and AEC. For negative controls, primary antibodies were substituted with an isotype matched IgG at the equivalent concentration of primary antibody.

### EXAMPLE 5

### Uptake of Dil-Ac-LDL

The presence of scavenger receptors for acetylated low-density lipoprotein (ac-LDL) on MEC was detected using 1,1 ′-dioctadecyl-1,3,3,3′,3′-tetramethylindocarbocyanine perchlorate acetylated LDL (DiI-Ac-LDL, Molecular Probes, Eugene, Oreg., USA). Confluent MEC cultured in 24 well plates on 0.2% gelatin were incubated with 15 μg/ml DiI-Ac-LDL (Collaborative Research, Bedford, Mass., USA) in culture medium for 4 hours at 37° C. in 5% CO₂/air according to the method Voyta et al. (1984). LDL uptake was examined using a Zeiss Axiovert 100 epifluorescence microscope, using optical bandpass filter set with an excitation of 545 nm and emission of 590 nm.

### EXAMPLE 6

### Endothelial Cell Proliferation Assay

The CellTitre 96 MTS tetrazolium-based bioassay (Promega, Madison, WT, USA), where the absorbance of the formazan product is directly proportional to the number of viable cells, was used to indirectly measure cell number after incubation of MEC with various mitogens. It was initially established that the MTS bioassay was linear from 0-2 ×10⁴ MEC/well. MEC in basal M199 medium (M199 medium containing 5% PCS, 2 mM glutamine, antibiotic/antimycotic mixture) were seeded in triplicate onto fibronectin (10 μg/ml) coated wells of 96 well plates at 2000 cells/well and allowed to attach the quiesce for 36 hours at 37° C. in 5% CO₂/air. Medium was replaced with the standard or complex M199 culture medium (day 0) with or without the following growth factors: 10 ng/ml vascular endothelial growth factor (VEGF₁₂₁;gift from Dr J Abrahams, Scios Inc, Sunnyvale, Calif., USA), 0.01-20 ng/ml VEGF₁₆₅ (R & D Systems, Minneapolis, Minn., USA) bFGF (0.01-10 ng/ml), 120 μg/ml EC growth factor (ECGF; Boehringer Mannheim) 50 ng/ml epidermal growth factor (EGF; R & D Systems), depending on the assay conditions being tested. In some experiments different concentrations of HS and FCS and combinations of both were tested and in other experiments various component of the complex medium were omitted. Cells were incubated at 37° C. in 5% CO₂/air for 6 or 10 days, with medium changes every 2 days. Twenty μl 3-(4,5-dimethylthiazol-2-yl)-5-(3-carboxymethoxyphenyl)-2-(4sulphophenyl)-2H-tetrazolium (MTS) was added to the cells and incubated 2 hours at 37° C. and absorbance measured at 490 nm using a plate reader (Emax, Molecular Devices, Sunnyvale, Calif., USA). Absorbance was also measured on day 0 and this value subtracted from all results to determine MEC growth over 6 days.

### EXAMPLE 7

### Mec Migration Assay

Early- (P3) and late- (P13) passage MEC were cultured in standard M199 culture medium in 0.2% gelatin-coated wells of a 24 well plate until confluent. The medium was changed to basal medium, the MEC monolayers wounded using a plastic pasteur pipette and photographed using a Zeiss inverted phase contrast microscope. Duplicate wells were incubated with or without VEGF₁₆₅ (10 ng/ml) or bFGF (5 ng/ml) in the presence and absence of 40 μg/ml rabbit anti human VEGF antibody (Gargett et al., 1999) or 50 μg/ml goat anti-human bFGF antibody (R & D Systems) respectively for 48 hours at 37° C. in 5% CO/air and the same field of view was rephotographed.

### EXAMPLE 8

### Statistical Analysis

Data were analysed by Student's t test using Excel software. P<0.05 was considered significant.

### EXAMPLE 9

### Isolation and Culture of Human Myometrial Microvascular Endothelial Cells

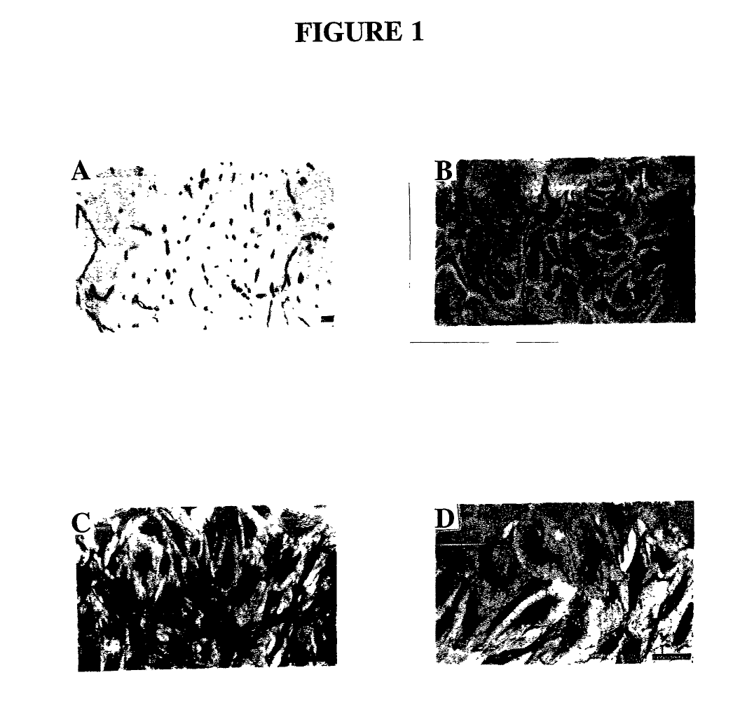

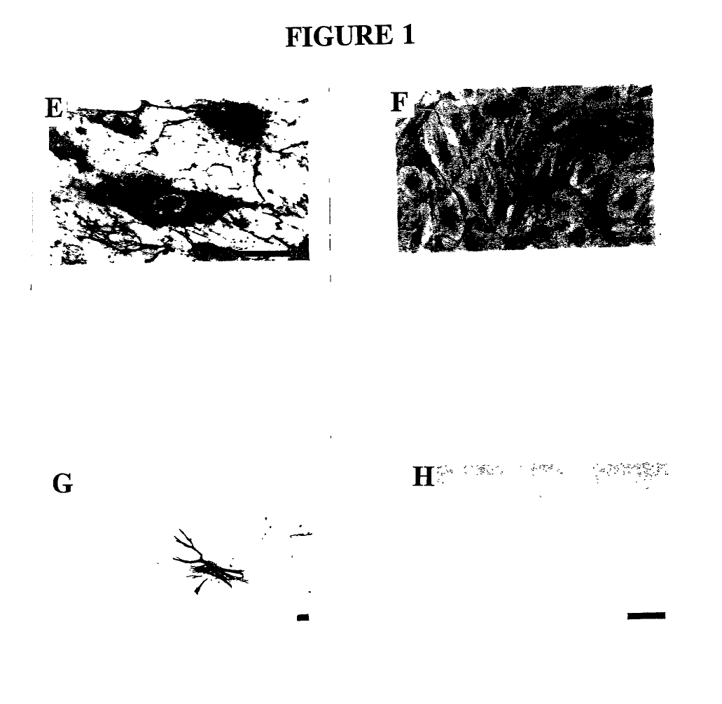

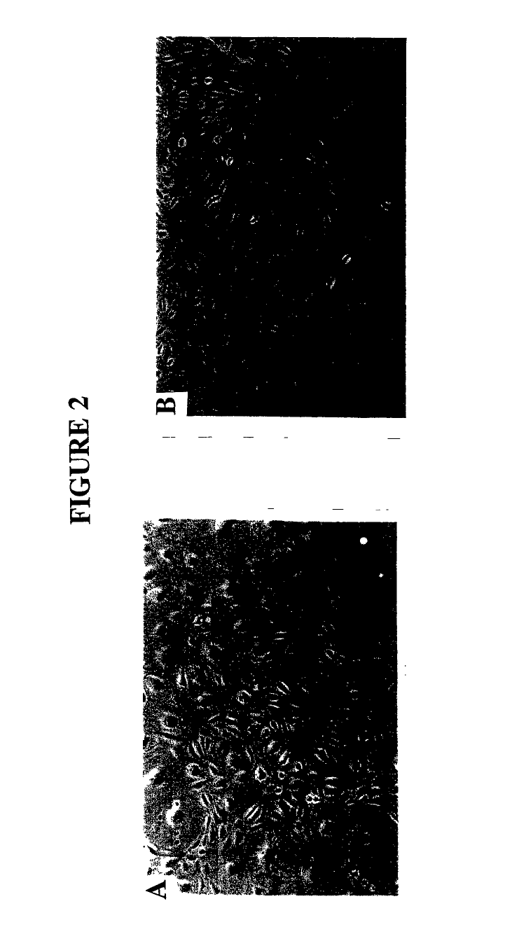

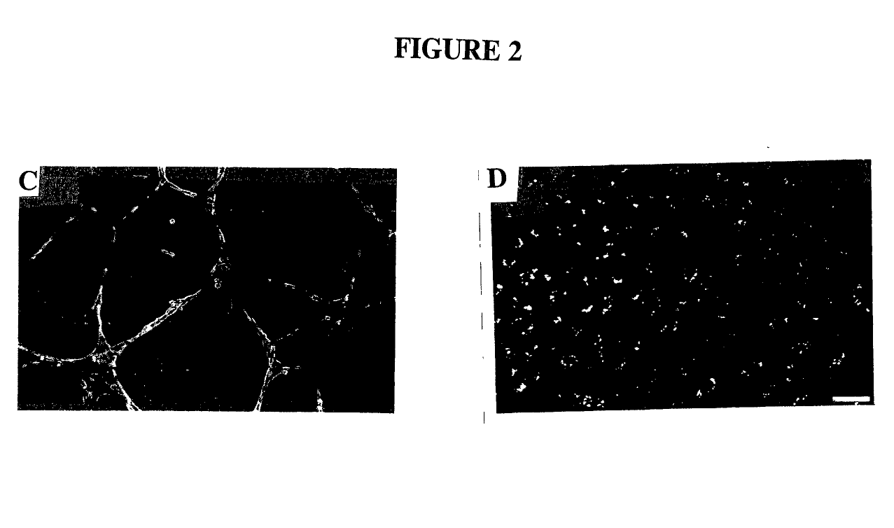

Human myometrium is a highly vascularized tissue as demonstrated by UEA binding to tissue sections (FIG. 1A). We have adapted the UEA-l-coated dynabead selection method of Jackson er al. (1990) to isolate and establish primary cultures of microvascular endothelial cells from human myometrium, which is primarily composed of smooth muscle cells and endothelial cells. Using this method our yields are approximately 10⁶ MEC per gram tissue and we usually process between 4-7 g. Primary cultures of MEC were readily established, although growth rates varied markedly between isolates, which reached confluence in 5 -10 days. We often found a further UEA-1-dynabead purification was required during early passage to produce >99.95% pure culture of MEC (FIGS. 1B-D, 1G and 2A, 2B). The attached dynabeads did not interfere with MEC growth or survival and were diluted out within one or two passages. While all MEC cultures demonstrated typical cobblestone morphology as viewed by phase contrast microscopy, the morphology or primary and P1 MEC was variable with respect to size and shape (FIGS. 1B, 2A). With increasing passage number, the morphology became very uniform (FIGS. 2B and 1C, 1D). At high passages (P14) MEC showed regular morphology but increased in size (FIG. 1D) and replicative senescence was reached by P16.

### EXAMPLE 10

### Characterisation of Human Myometrial Mec

Cultured myometrial MEC displayed characteristic features of endothelial cells. At all passages examined (P1-P14), MEC bound UEA-1 lectin (FIG. 1B-D), expressed factor VIII-related antigen in Weibel-Palade bodies (FIG. 1E), CD31 in the perinuclear region and at sites of cell-cell contact (FIG. 1F), took up DiI-Ac-LDL via the scavenger LDL receptor (FIG. 2D) and formed capillary-like tubes on the basement membrane matrix, Matrigel (FIG. 2C). Myometrial MEC did not immunostain with cytokeratin antibodies (results not shown) or express the smooth muscle cell marker, α-smooth muscle actin, as seen by the lack of staining of the MEC in the background of FIG. 1G.

### EXAMPLE 11

### Growth Requirements and Characteristics of Human Myometrial Mec

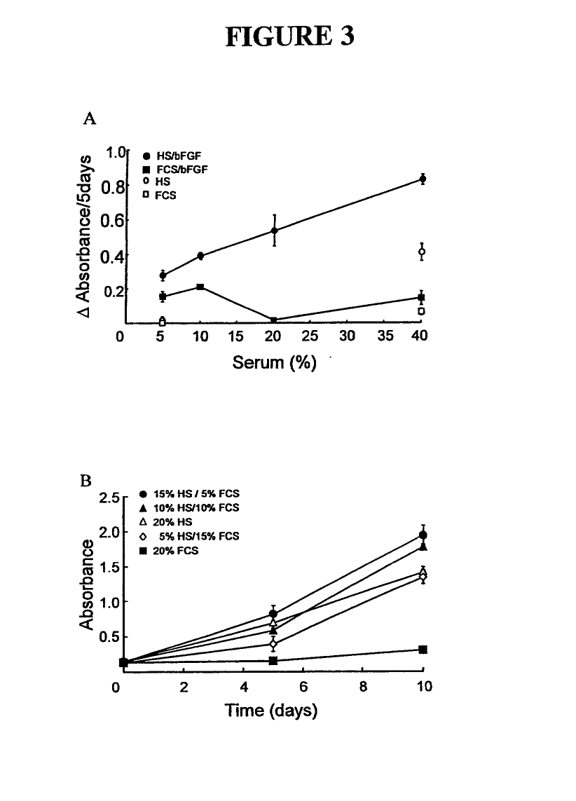

FIG. 3 shows that myometrial MEC had an absolute requirement for human serum for growth stimulated by either bFGF (FIG. 3A) or VEGF (results not shown), and that this effect was dose-dependent. Even in the absence of growth factor, 40% HS stimulated significant MEC proliferation (P<0.03) (FIG. 3A). While myometrial MEC survived in FCS containing medium, neither bFGF (FIG. 3A) nor VEGF (results not shown) induced significant MEC proliferation, even at high FCS concentrations. However, both bFGF (FIG. 3B) and VEGF (results not shown) stimulated even greater levels of MEC proliferation when combinations of HS and FCS were present in the medium (FIG. 3B). the optimal combination was 15% HS and 5% FCS (FIG. 3B).

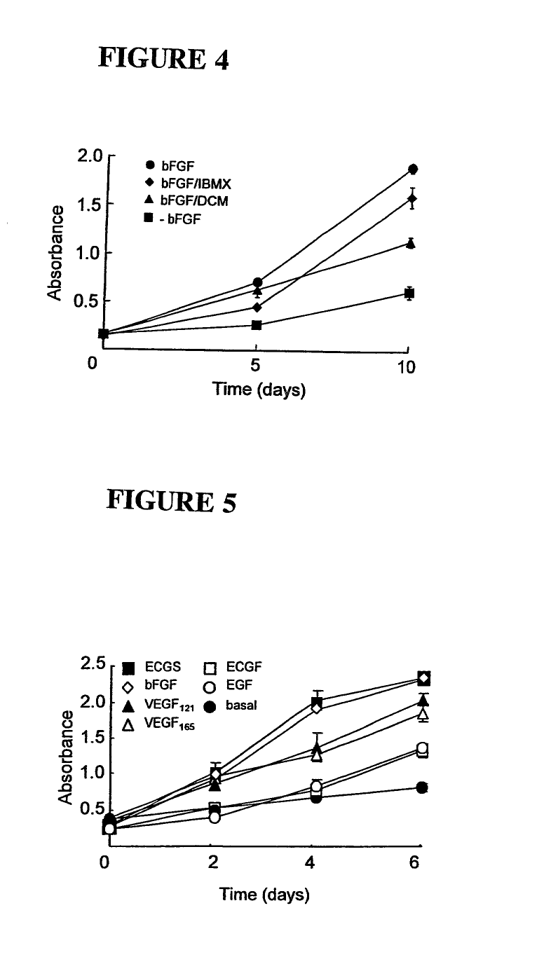

Agents which increase intracellular levels of adenosine 3′,5′-cyclic monophosphate (cAMP) are commonly used in the mediumn for culture of MEC to stimulate their growth (Karasek, 1989). The effect of a cAMP phosphodiesterase inhibitor, 3-isobutyl-1-methyl xanthine (IBMX 330 μM) and a membrane permanent cAMP analogue, dibutyryl cAMP (N-6, 2 ′-O-dibutyryl adenosine 3′,5′-cyclic monophosphate, 50 μM, Sigma) was examined on bFGF-stimnulated MEC proliferation. FIG. 4 shows that bFGF stimulated significantly greater proliferation of myometrial MEC over 10 days in the absence of either IBMX or dibutyryl cAMP (P<0.05). After 5 days greater growth was observed in the absence of dibutyryl cAMP (P<0.02) but not IBMX (P=0.06) (FIG. 4). Hydrocortisone had no significant effect on bFGF-induced myometrial MEC proliferation in the presence or absence of either IBMX or dibuytryl cAMP (results not shown).

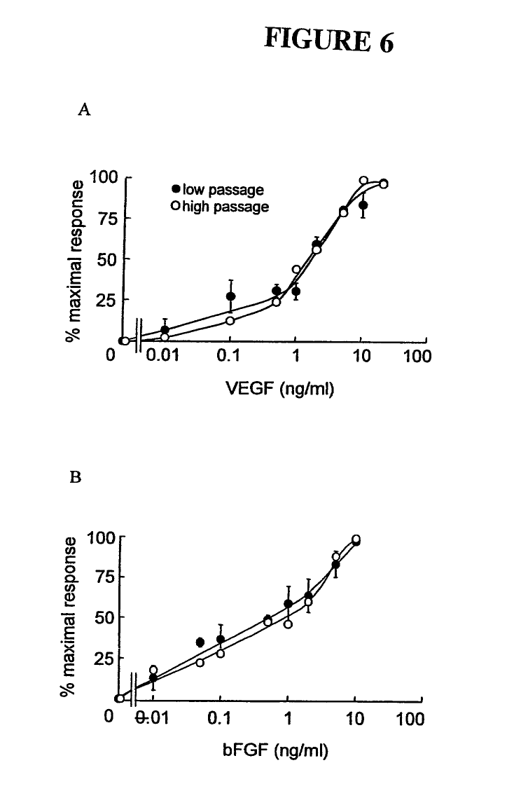

The responsiveness of human myometrial MEC to a range of growth factors, known to induce proliferation of EC derived from other tissues, was determined by stimulating MEC with maximal concentrations of growth factors for 6 days. FIG. 5 shows that ECGS and bFGF were equally effective and produced maximal stimulation of MEC proliferation, followed by the 2 VEGF isoforms, VEGF₁₂₁, and VEGF₁₆₅. ECGF and EGF were weakly mitogenic for myometrial MEC. Both VEGF₁₆₅ and bFGF stimulated myometrial MEC proliferation in a dose dependent manner over the concentration range 0.01 -20 ng/ml (FIG. 6). The dose response curves obtained from high and low passage MEC were very similar for bFGF and VEGF when reported as the mean percentage of maximal responses (FIG. 6). The approximately EC₅₀ for VEGF was 1.5 and 1.6 ng/ml for early and late passage MEC respectively and for bFGF, 0.6 and 0.9 ng/ml. The difference in absorbance between maximal and minimal response measured at day 6 was significantly greater for high passage MEC compared to low passage MEC for bFGF:0.50 ±0.07 (n=3) versus 0.82 (n=2) respectively (P=0.044), but not for VEGF: 0.51 ±0.09 (n=3) versus 0.70 (n=2) respectively (P=0.33). These results indicate that late passage MEC proliferate at a greater rate in response to bFGF compared to early passage MEC.

### EXAMPLE 11

### Migration Responses of Myometrial Mec

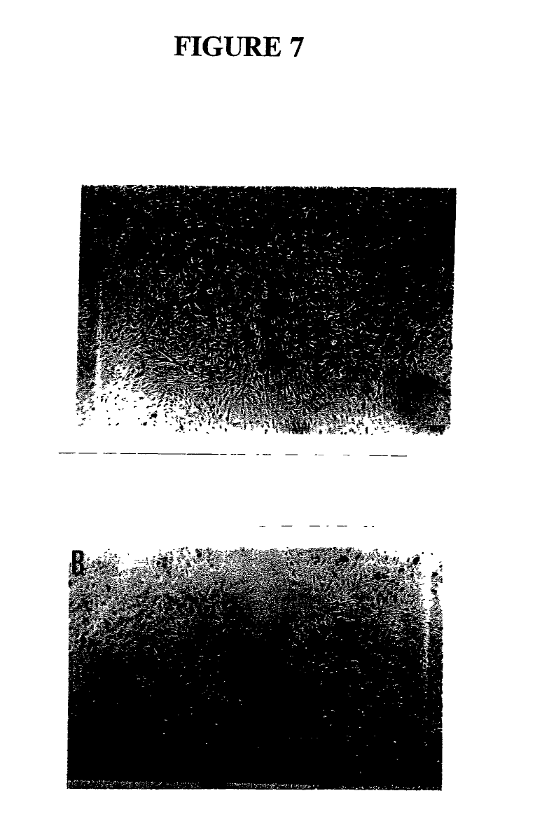

A characteristic feature of EC is their ability to migrate in response to certain growth factors (Goodman et al., 1985; Ferrara and DavisSmyth, 1997). The migration of early and late passage myometrial MEC in response to bFGF and VEGF was examined using the monolayer wound assay (Goodman el al., 1985) in the presence and absence of neutralising bFGF and VEGF antibodies. After a 48 hour incubation in basal medium in the presence of bFGF or VEGF, both high and low passage MEC migrated into the denuded area to a comparable extent, and this migration was completely inhibited by the respective neutralising antibodies to each growth factor (FIG. 7, Table 2).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

Bibliography

Abbott, S. E., Kaul A., Stevens, C. R. et al. (1996) Synovial microvascular endothelial cell isolation and culture. In Biclmell R (Ed) Endothelial cel culture. Cambridge University Press, Cambridge, UK, pp. 115-132.

Abberton, K. M., Taylor, N. H., Healy, D. L. et al. (1999) Hum. Reprod., 14:1072-1079.

Auerbach, R., Alby, L., Morrissey, L. W. et al. (1985) Microvasc. Res. 29:401-411.

Bicknell, R. (1996) Introduction to the endothelial cell. In Bicknell R (Ed) Endothelial cell culture. Cambridge University Press, Cambridge, UK, pp. 1-6.

Carley, W. W., Niedbala, M. J. and Gerritsen, M. E. (1992) Am. J. resp. Cell Mol. Biol. 7:620-630.

Chung-Welch, N., Patton, W. F., Shepro, D. et al. (1997) Microvasc. Res. 54:121-134.

DeLisser, H. M., Christofidou-Solomidou, M., Strieter, R. M. et al. (1997) Am. J. Pathol. 151:671-677.

Fawcett, J., Harris, A. L. and Bicknell, R. (1991) Biochem. Biophys. Res. Commun. 174:903-908.

Ferrara, N. and DavisSmyth, T. (1997) Endocrine. Rev. 18:4-25.

Gallery, E. D. M., Rowe, J., Schrieber, L. et al. (1991) Am. J. Obster. Gynecol. 165:191-16.

Gargett, C. E., Lederman, F. L., Lau, T. M. er al. (1999) Lack of correlation between vascular endothelial growth factor production and endothelial cell proliferation in the human endometriuin. Hum. Reprod., (in press)

Garlanda, C. and Dejana, E. (1997) Arterioscler. Thromb. Vasc. Biol. 17:1193-1202.

Goodger (MacPherson), A. M. and Rogers, P. A. W. (1994) Hum. Reproduc. 9:399-405.

Goodrnan, S. L., Vollmers, H. P. and Birchmeier, W. (1985) Cell 41:1029-1038.

Grimnwood, J., Bicknell, R. and Rees, M. C. P. (1995) Hum. Reprod. 10.2142-2148.

Haraldsen, G., Rugtveit, J., Kvale, D. et at. (1995) Gut 37:225-234.

Hewett, P. W. and Murray, J. C. (1993) In Vitro Cell Dev. Biol. 29:823-830.

Iruela-Arispe, M. L., Rodriguez-Manzaneque, J. C. and Abu-Jawdeh, G. (1999) Microcirc. 6:127-140.

Jackson, C. J., Garbett, P. K., Nissen, B. et al. (1990) J. Cell Sci. 95:257-262.

Jaffe, E. A., Nachman, R. L., Becker, C. G. et al. (1973) J. Clin. Invest. 52:2745-2756.

Karasek, M. A. (1989) J. Invest. Dennatol. 93:33S-38S.

Kraling, B. M. and Bischoff, J. (1998) In Virto Cell Dev. Biol.33:308-315.

Krupinski, J. (1991) Molec. Cell. Biochem. 104.73-79.

Kumar, S., West, D. C. and Ager, A. (1987) Differenlanon 36:57-70.

McCarthy, S. A., Kuzu, I., Gatter, K. C. et al. (1991) TIPS 12:462-467.

McDouall, R. M., Yacoub, M. and Rose, M. L. (1996) Microvasc. Res. 51:137-152.

Newman, P. J. (1997) J. Clin. Invest. 99:3-8.

Nishida, M., Carley, W. C., Gerritsen, M. E. et al. (1993) Am. J. PhysioL 264:H639-H652.

Noe, M., Kunz, G., Herbertz, M. et al. (1999) Hum. Reprod. 14:190-197.

Richard, L., Velasco, P. and Detmar, M. (1998) Exp. Cell Res. 240:1-6.

Risau, W. (1997) Nature 368.671-674.

Sanyal, A. J. and Mirshahi, F. (1998) Lab. Invest. 78:1469-1470.

Su, T. and Gilles, M. C. (1992) Invest. Ophthalmol. Vis. Sci. 33:2809-2813.

Tschugguel, w., Zhegu, Z., Schneeberger, C. et al. (1997) J. Vasc. Res. 34:281-288.

Voyta, J. C., Via, D. P., Butterfield, C. E. et al. (1984) J. Cell Biol. 99:2034-2040.

TABLE 2

| | Early Passage | Late Passage |
|---|---|---|
| Condition | MEC (P4) | MEC (P12) |
| Control | + | + |
| VEGF | +++ | +++ |
| VEGF + anti-VEGF Ab | +/− | +/− |
| bFGF | +++ | ++ |
| bFGF + anti-bFGF Ab | +/− | +/− |
| Anti-VEGF + anti-bFGF Abs | +/− | +/− |

The migration of MEC into a denuded area of the culture well following wounding of the monolayer was evaluated semi-quantitatively, represented by +/−, very low; +, low; ++, medium; +++, high coverage of the denuded area.

1. A method for the in vitro culturing of microvascular endothelial cells, said method comprising culturing an enriched population of microvascular endothelial cells in the presence of an effective amount of human serum or functional derivative or equivalent thereof for a time and under conditions sufficient to support the growth of said microvascular endothelial cells.

2. A method for the in vitro culturing of microvascular endothelial cells, said method comprising culturing an enriched population of microvascular endothelial cells in the presence of an effective amount of human serum or functional derivative or equivalent thereof and a mitogen or functional derivative or equivalent thereof for a time and under conditions sufficient to support the growth of said microvascular endothelial cells.

3. The method according to claim 1 or 2 wherein said microvascular endothelial cells are myometrial microvascular endothelial cells.

4. The method according to claim 3 wherein said myometrial microvascular endothelial cells are human myometrial microvascular endothelial cells.

5. the method according to claim 1 or 2 wherein said enriched population of microvascular endothelial cells is an at least 99% pure population of microvascular endothelial cells.

6. The method according to claim 5 wherein said microvascular endothelial cells are myometrial microvascular endothelial cells.

7. The method according to claim 6 wherein said myometrial microvascular endothelial cells are human myometrial microvascular endothelial cells.

8. The method according to claim 1 or 2 wherein said enriched population of microvascutar endothelial cells are obtained by enriching a starting cellular population comprising microvascular endothelial cells, which enrichment comprises the steps of:
(i) selecting for microvascular endothelial cells from said starting population;
(ii) pre-culturing the cells selected in accordance with step (i) to 60-80% confluence; and
(iii) selecting for microvascular cndotielial cells from the 60-80% confluent cell population of step (ii).

9. The method according to claim 8 wherein said microvascular endothelial cells are myometrial microvascular endothelial cells.

10. The method according to claim 9 wherein said myometrial inicrovascular erndothelial cells are huinan myometrial microvascular endothelial cells.

11. The method according to claim 10 wherein said starting cellular population of hunman myometrial microvascular endotlhelial cells is a population of enzymatically digested, tissue sample derived cells.

12. The iethod according to claim 2 wherein said mitogen is bFGF.

13. The method according to claim 12 wherein said human serum is utilised at 20% v/v and said bFGF is utilised at 5 ng/ml.

14. A method for the in vitro culturing of microvascular endothelial cells, said method comprising culturing an enriched population of microvascular endothelial cells in the presence of an effective amount of human serum or fnctional derivative or equivalent thereof together with fetal calf serum and a mitogen or functional derivative or equivalent thereof for a time and under conditions sufficient to support the growth of said microvascular endothelial cells.

15. The method according to claim 14 wherein said human serum is utilised together with said fetal calf serum at a ratio of not less than 3:1 and said mitogen is either bFGF or VEGF.

16. The method according to claim 15 wherein said human serum is utilised at 15% v/v and said fetal calf serum is utilised at 5% v/v.

17. The method according to claim 16 wherein said culturing is additionally performed in the presence of an effective amount of heparin.

18. The method according to claim 17 wherein said mitogen is 5 ng/ml bFGF and said heparin is utilised at 0.1 mg/ml.

19. The method according to any one of claims 14-18 wherein said microvascular endothelial cells are myometrial microvascular endothelial cells.

20. The method according to claim 19 wherein said myometrial microvascular-endothelial cells are human myometrial microvascular endothelial cells.

21. The method according to claim 20 wherein said enriched population of microvascular endothelial cells are obtained by enriching a starting cellular population comprising microvascular endothelial cells, which enrichment comprises the steps of:
(i) selecting for myometrial microvascular endothelial cells from said starting population;
(ii) pre-culturing the cells selected in accordance with step (i) to 60-80% confluence; and
(iii) selecting for microvascular endothelial cells from the 60-80% confluent cell population of step (ii).

22. The method according to claim 21 wherein said starting ccllular population of human myometrial microvascular endothelial cells is a population of enzymatically digested, tissue sample derived cells.

23. A method for the in virro culturing of microvasculwa endothelial cells, said method comprising culturing an enriched population of microvascular endothelial cells in the presence of an effective amount of human serum or functional derivative or equivalent thereof and a mitogen or fuctional derivative or equivalent thereof for a time and under conditions sufficient to support the growth of said microvascular endotheclial cells wherein said culture does not comprise cAMP or functional derivative or equivalent thereof or corticosteroids or functional derivative or equivalent thereof.

24. The method according to claim 23 wherein said microvascular endothelial cells are myometrial microvascular endothelial cells.

25. The method according to claim 24 wherein said myometrial microvascular endothelial cells are human myometrial microvascular endothelial cells.

26. A method of producing a microvascular endothelial cell culture said method comprising culturing an enriched population of microvascular endothelial cells in the presence of an effective amount of human serum or functional derivative or equivalent thereof and a mitogen or functional derivative or equivalent thereof for a time and under conditions sufficient to support the growth of said microvascular endothelial cells.

27. The method according to claim 26 wherein said microvascular endothelial cells are myometrial microvascular endothelial cells.

28. The method according to claim 27 wherein said myometrial microvascular endothelial cells are human myometrial microvascular endothelial cells.

29. The method according to any one of claims 23-28 wherein said human serum is utilised together with fetal calf serum at a ratio of not less than 3:1 and said mitogen is either bFGF or VEGF.

30. The method according to claim 29 wherein said human serum is utilised at 15% v/v and said fetal calf serum is utilised at 5% v/v.

31. The method according to claim 30 wherein said culturing is additionally performed in the presence of an effective amount of heparin.

32. The method according to claim 31 wherein said mitogen is 5 ng/ml bFGF and said heparin is utilised at 0.1 mg/ml.

33. The method according to any one of claims 23-28 wherein said human serum is utilised at 20% v/v and said bFGF is utilised at 5 ng/ml.

34. The method according to claim 24 or 27 wherein said enriched population of myometrial microvascular endothelial cells are obtained by enriching a starting cellular population comprising microvascular endothelial cells, which enrichment comprises the steps of:
(i) selecting for myometrial microvascular endothelial cells from said starting population;
(ii) pre-culturing the cells selected in accordance with step (i) to 60-80% confluence; and
(iii) selecting for microvascular endothelial cells from the 60-80% confluent cell population of step (ii).

35. The method according to claim 34 wherein said starting cellular population of human myometrial microvascular endothelial cells is a population of enzymatically digested, tissue sample derived cells.

36. A method for the therapeutic and/or prophylactic treatment of an individual said method comprising the use of microvascular endothelial cells generated in accordance with the method of any one of claims 1, 2, 14, 23 or 26.

37. A diagnostic procedure comprising the use of microvascular endothelial cells generated in accordance with the method of any one of claims 1, 2, 14, 23 or 26.

38. A microvascular endothelial cells produced in accordance with the method of any one of claims 1, 2, 14, 23 or 26.

39. A microvascular endothelial cell culture produced in accordance with the method of any one of claims 1, 2, 14, 23 or 26.
